(19) United States
(12) Patent Application Publication
(10) Pub. No.: US 2004/0092846 A1
(43) Pub. Date: May 13, 2004

(54) Graphical user interface and voice-guided protocol for an auscultatory diagnostic decision support system

(76) Inventor: Raymond L. Watrous, Belle Mead, NJ (US)

Correspondence Address: RATNERPRESTIA, P O BOX 980, VALLEY FORGE, PA, 19482-0980, US

(21) Appl. No.: 10/698,646

(22) Filed: Oct. 31, 2003

Related U.S. Application Data
(60) Provisional application No. 60/422,645, filed on Oct. 31, 2002.

(51) Int. Cl. A61B 7/00

(52) U.S. Cl. 600/586

(57) ABSTRACT

The present invention relates to an apparatus and method for determining an auscultatory diagnostic decision. Auscultation of the heart is a well-defined and standard component of physical examinations of patients, however it is a difficult procedure particularly because a stethoscope transfers only a small fraction of the acoustic signal at the chest surface to the listener's ears and filters the cardiac acoustic signal in the process. The system assists listeners by implementing a graphical user interface and voice guided protocol to record data and analyze results for the presence of heart sounds and murmurs. The results are identified in terms of standard clinical auscultatory findings which may then be used to make diagnostic and referral decisions.

This application is related to and claims the benefit of U.S. Provisional Application No. 60/422,645 entitled GRAPHICAL USER INTERFACE AND VOICE-GUIDED PROTOCOL FOR AN AUSCULTATORY DIAGNOSTIC DECISION SUPPORT SYSTEM filed on Oct. 31, 2002.

### FIELD OF THE INVENTION

The present invention relates to the field of systems and methods for automatically analyzing heart sounds from a patient following a defined protocol, specifically user interfaces for heart sound recording and analyzing systems.

### BACKGROUND OF THE INVENTION

Auscultation of the heart is a well-defined and standard component of the physical examinations of patients. It is typically performed with a commercially available stethoscope. Physicians perform auscultation by listening to heart sounds desirably, in sequence, at a set of well-defined sites on the chest surfaces. These sites are typically defined with reference to anatomical landmarks, such as the second intercostal space on the left, etc. They may also be defined based on the heart valve preferentially heard at that location (i.e., aortic, pulmonic, etc.). Additionally, auscultation can be carried out with the patient in different postures, or while executing various maneuvers that are designed to enhance or suppress certain murmurs.

Auscultation of the heart is a difficult procedure, involving significant training. Stethoscopes transfer only a small fraction of the acoustic signal at the chest surface to the listener's ears and filter the cardiac acoustic signal in the process. A significant portion of the signal energy in heart sounds is at frequencies below the frequency range of human hearing, and this situation only tends to worsen with increased age of the listener. Thus, as a physician's auscultatory skill increases, his hearing may still limit his ability.

Also, auscultation relies on determining the correct sequence of brief events that are closely spaced in time, a determination that may be difficult for human listeners. Furthermore, auscultation relies on determining the correspondence of the primary heart sounds with the length of the systolic and diastolic phase of the heart. This becomes more difficult when the systolic and diastolic intervals are more equal, which typically occurs at elevated heart rates.

The practice and teaching of the clinical skill of auscultation of the heart has declined among physicians. Learning auscultation is complicated by the reliance of diagnostic instructional manuals that rely on subjective descriptions of heart sounds, which require much practice to appreciate. Recent tests have demonstrated that many physicians can reliably identify only a small number of standard heart sounds and murmurs, as described by Burdick et al., in “Physical Diagnosis Skills of Physicians in Training: A Focused Assessment,” Acad. Emerg. Med., 2(7), pp. 622-29, July 1995; Mangione et al., in “Cardiac Auscultatory Skills of Internal Medicine and Family Practice Trainees: A Comparison of Diagnostic Proficiency,” Journal of the American Medical Association, 278(9), pp. 717-22, September 1997; and Gracely et al., in the Teaching and Practice of Cardiac Auscultation During Internal Medicine and Cardiology Training: A Nationwide Survey,” Annals of Internal Medicine, 119(1), pp. 47-54, July 1997. Consequently, serious heart murmurs in many patients may go undetected by physicians relying on standard auscultation technique.

This decline in auscultation skills has led to an over-reliance on echocardiography, resulting in a large number of unnecessary and expensive diagnostic studies. Thus, economic factors have also lead to an interest in improving auscultatory screening procedures. One approach that has generated interest is the recording of heart sounds for automated analysis to assist the physician in making a diagnosis.

Because the site of the heart sound recording has clinical significance, it is desirable that there be a correct association between each particular recorded signal and the corresponding recording site for accurate and complete analysis. Accordingly, while systems may require the user to input the site for each signal, it is desirable and highly advantageous to have a system where a user follows a predetermined sequence and is guided through that sequence. Additionally, a system with a more user friendly interface may desirably allow preliminary tests to be performed by less skilled personnel. For example, when using such a system, a nurse may record the data, leaving the doctor to analyze the results, thereby saving the doctor's time to assist other patients.

### SUMMARY OF THE INVENTION

The context for this invention is a diagnostic decision support system and method for auscultation of the heart. The system assists listeners by implementing a graphical user interface in combination with a voice-guided protocol. Heart sounds are recorded from well-defined and standard positions on the chest, using a noninvasive, passive acoustic sensor, such as a commercially available electronic stethoscope. The recorded heart signals are analyzed for the presence of heart sounds and murmurs, which are then desirably identified, characterized, and described in terms of standard clinical auscultatory findings. These findings may be used by a physician to make diagnostic and referral decisions.

An exemplary embodiment of the present invention provides a predetermined protocol corresponding to a plurality of recording locations on a patient. The user is instructed to follow the predetermined protocol for recording a plurality of heart sounds at a plurality of recording locations using at least one of a voice guided protocol and/or a graphical user interface. Heart sounds are detected and recorded according to the predetermined protocol. Finally, the heart sounds are analyzed to determine the auscultatory findings which support a diagnostic decision by the physician.

Another exemplary embodiment of the present invention is an auscultatory diagnostic decision support system. The exemplary auscultatory diagnostic decision support system includes a cardiac acoustic sensor to produce a heart sound signal, a heart sound analysis device adapted to receive and analyze the heart sound signal, and a display device which includes a graphical user interface to guide a user through a predetermined protocol.

A further exemplary embodiment of the present invention is a user interface for an auscultatory diagnostic decision support system which includes a graphical user interface. The exemplary graphical user interface includes a visual representation of at least the anterior thorax to guide the user, a plurality of positional markers to pinpoint a plurality of auscultatory measurement locations on the anterior thorax, a visual presentation of a measured acoustic signal corresponding to each auscultatory measurement location, and a speaker to provide a voice guided protocol, which includes a series of audio prompts to guide the user through the voice guided protocol.

The present invention may also be applicable to signal acquisition and conditioning, or filtering, these signals to produce an enhanced phonocardiogram (PCG), by which relevant signal characteristics can be enhanced and readily viewed.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawing are the following figures:

FIG. 1 is a block diagram of an exemplary auscultation diagnostic decision support system according to the present invention.

FIG. 2 is a flow chart illustrating an exemplary method of using the system of FIG. 1 according to the present invention.

FIG. 3 is a schematic drawing of an exemplary graphical user interface display illustrating an exemplary auscultation acquisition protocol display.

FIG. 4 is a schematic drawing of an exemplary graphical recording position map of the exemplary graphical user interface display of FIG. 3.

FIGS. 5A-5D are schematic drawings of an exemplary graphical recording position map illustrating four exemplary recording locations of exemplary graphical recording positions.

FIG. 6 is a schematic drawing of an exemplary graphical user interface display illustrating an exemplary auscultation analysis display.

### DETAILED DESCRIPTION OF THE INVENTION

The present invention involves a system and method for determining an auscultatory diagnostic decision. A voice guided protocol and a graphical user interface are desirably combined to assist with the recordation, analysis, and reporting of data.

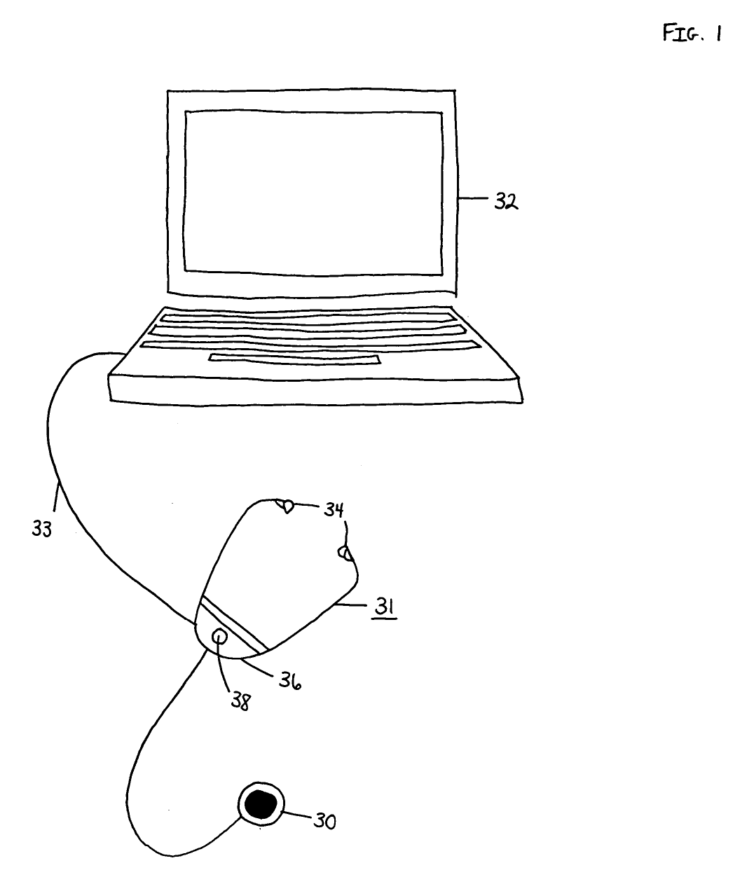

FIG. 1, shows an exemplary embodiment of the present invention. This exemplary auscultation diagnosis support system includes electronic stethoscope 31 and general purpose computer 32. Signals representing heart sounds detected by electronic stethoscope 31 may be transmitted to general purpose computer 32 over transmission line 33, or may be transmitted via an infrared or wireless broadcast signal.

In the exemplary auscultatory diagnostic decision support system illustrated in FIG. 1, electronic stethoscope 31 is a commercially available electronic stethoscope which includes cardiac acoustic sensor 30, earpieces 34, amplification and filtering circuitry 36, and amplification control interface 38. During operation of this exemplary auscultatory diagnostic decision support system, electronic stethoscope 31 is used to detect heart sounds and transmit these sounds as an electrical signal to general purpose computer 32 for analysis and display. If the electrical heart sound signal is transmitted to general purpose computer 32 via infrared or wireless means, electronic stethoscope 31 may include an infrared source, such as an LED, or a wireless antenna (not shown) to propagate the signal.

Cardiac acoustic sensor 30 detects the hearts sounds and converts these sounds into electrical signals. These electronic signals are then amplified and/or filtered by amplification and filtering circuitry 36. The characteristics of cardiac acoustic sensor 30 and amplification and filtering circuitry 36 vary between different electronic stethoscopes, leading to heart sound signals having different bandwidth and intensity characteristics. It is desirable to account for these characteristics, during analysis of the heart sound signals by general purpose computer 32. The amplification level and possibly filtering characteristics of amplification and filtering circuitry 36 may be controlled by the user with amplification control interface 38, based on the signal heard in earpieces 34. It is noted that, although amplification and filtering circuitry 36 and amplification control interface 38 are shown as located in the crux of the electronic stethoscope, one or both of these elements may be located in the sensor head along with cardiac acoustic sensor 30, or they may be integral to general purpose computer 32.

Earpieces 34 allow the user to simultaneously listen to the heart sounds to monitor the sounds for volume and clarity. The user may also listen to the heart sounds to determine auscultatory findings as in a non-automated auscultatory examination. These determinations may then be augmented by the auscultatory findings of the exemplary system to provide improved diagnostic accuracy.

It is also contemplated that electronic stethoscope 31 may be replaced by a special purpose device including a self contained cardiac acoustic sensor with associated circuitry and a separate speaker. The speaker of general purpose computer 32 may be used for this purpose.

The heart sound analysis and display of both the raw heart sound signals and the auscultatory findings may desirably be performed by computer program instructions that control general purpose computer 32 where the computer program instructions reside on a computer-readable carrier such as a magnetic or optical disk or a radio-frequency or audio-frequency carrier wave. Although general purpose computer 32 is shown in FIG. 1 as a laptop computer, it may alternatively be a desktop computer or may include a local terminal with a display connected to a remote server where the analysis may occur. General purpose computer 32 is desirably one such that the system has sound input functions with sufficient bandwidth and spectral response to enable all of the desired features of the heart sound signals to be received for analysis.

It is also contemplated that the heart sound analysis could be performed by special purpose heart sound signal processing circuitry, possibly embodied in an application-specific integrated circuit (ASIC), instead of by a general purpose computer. Alternatively, preprocessing of the heart sound signals may be performed by signal processing circuitry and further analysis carried out by cardiac signal diagnostic software instructing the general purpose computer.

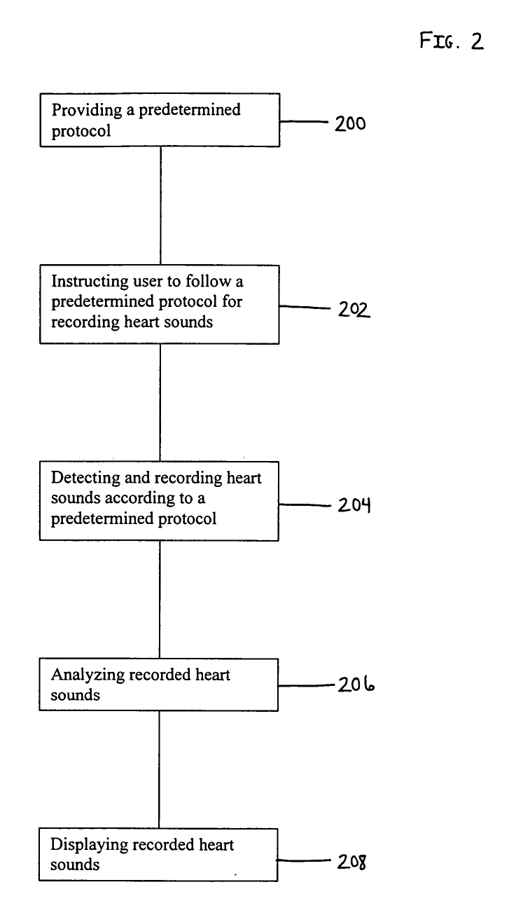

FIG. 2, shows an exemplary method for determining an auscultatory diagnostic decision using the exemplary system of FIG. 1. A predetermined protocol is provided, step 200, which corresponds with a selected plurality of recording locations on the patient. Desirably, this predetermined protocol includes the order of the desired recording locations, the time for recording data at each location, and any information regarding the posture of the patient and/or any dynamic auscultatory maneuvers to be performed at each recording location. Exemplary recording postures may include sitting, standing, squatting, and reclining.

The user is instructed to follow the predetermined protocol for recording heart sounds at each of the desired recording locations, step 202. This instruction is desirably supplied by both a voice guided protocol and a graphical user interface, which lead the user through the steps of the auscultatory procedure. In this way, the user is instructed to follow the predetermined protocol for recording heart sounds at the selected recording locations with both auditory and visual prompts. Thus, the detecting and recording of the heart sounds, step 204, desirably occur according to the predetermined protocol. This step of detecting and recording the heart sounds may further include prompting the user to re-record heart sounds from one of the recording locations if the heart sound is determined to include an error, such as the presence of an unacceptable noise (either due to background sounds or electronic noise within the system), a signal clipping, or a loss of signal. Additionally, it may be desirable for the user to be allowed to override the predetermined protocol, particularly to allow re-recording of data at a recording location where the user believes that the heart sound may have included an error.

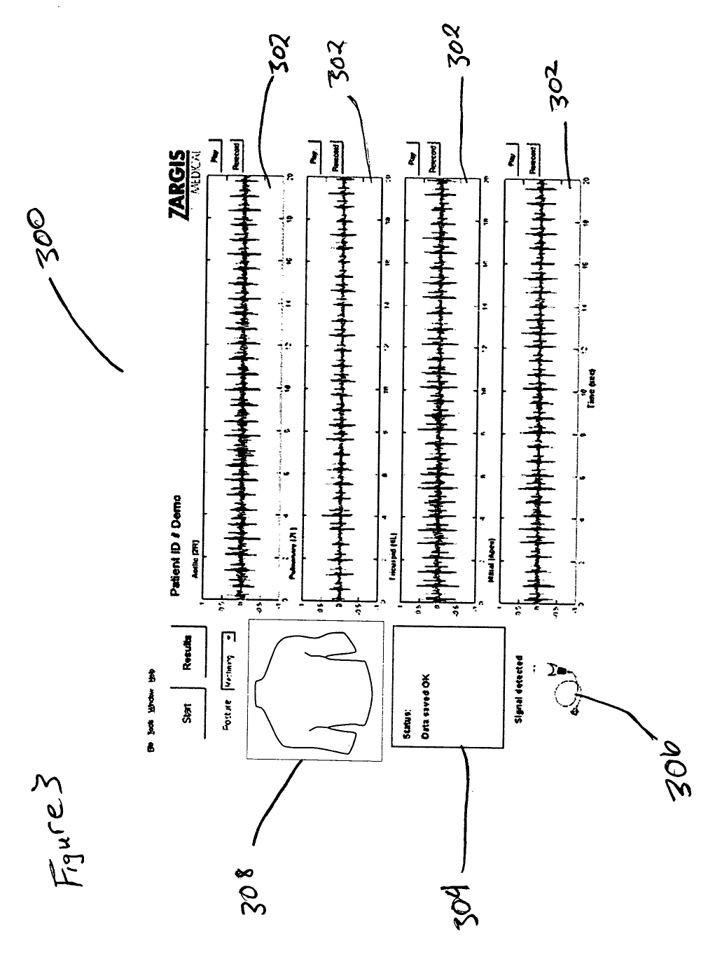

FIG. 3 illustrates exemplary testing procedure screen display 300 of the graphical user interface, which may be used to provide visual prompts. Exemplary testing procedure screen display 300 may include several sections, such as heart sound signal traces 302, status indicator 304, signal detection icon 306, and graphical recording position map 308. The organization of the sections within testing procedure screen display 300, as shown in FIG. 3, is merely illustrative. Other organizations may be used, or the various desired sections may alternatively be arranged in separate windows that may be organized or tiled by the user as desired. Additionally, the possible sections of the testing procedure screen display may be provided as entries in a pull down, or other, menu from which the user may select desired sections to view.

Desirably, testing procedure screen display 300 may be organized to include one heart sound trace 302 for each recording position in the predetermined protocol. These heart sound traces are desirably arranged vertically in sequential order of the predetermined protocol and may be displayed one at a time following the recording of the heart sounds for each recording position. Alternatively, all of the heart sound traces may be displayed at the end of the recording sequence.

Status indicator 304 may be displayed as a text box, as shown in FIG. 3, or may be displayed as an indicator bar and/or a set of predetermined icons, selected to indicate the current status and progress of the auscultatory test procedure. Signal detection icon 306 may include text and/or an icon (both are shown in FIG. 3) to indicate that the heart sound signal is being properly received by the recording and analysis device.

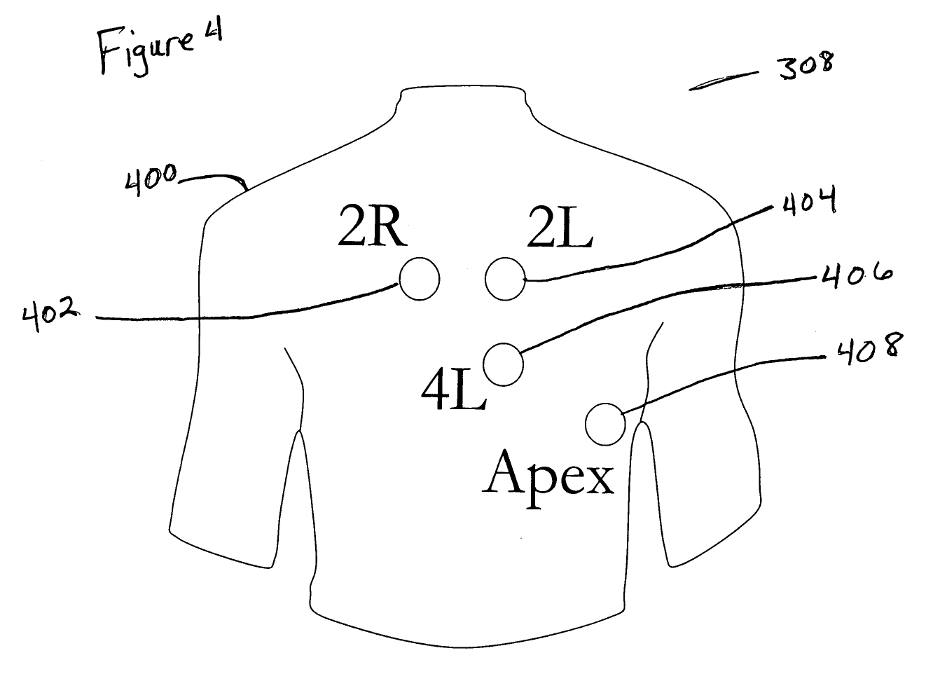

As shown in FIG. 4, graphical recording position map 308 of exemplary testing procedure screen display 300 of the graphical user interface may desirably include a visual representation of the desired body portion of a patient, such as anterior thorax 400, to guide the user through the predetermined protocol. Depending on the auscultatory tests to be conducted, the body portion visually represented may include the posterior thorax, the anterior abdomen, and/or the posterior abdomen, as well as the exemplary anterior thorax 400 shown in FIG. 4.

Target areas for sensor placement (i.e. the recording locations) may be desirably identified on the visual body part representation by positional markers, such as exemplary positional markers 402, 404, 406, and 408 shown in FIG. 4. These positional markers are desirably solid or translucent symbols, such as small circles, triangles, or other symbols, which pinpoint desired placements of the cardiac acoustic sensor 30 on the anterior thorax 400. Text indicating the designation of the anatomical locations (2R, 2L, 4L, and Apex, for example) may be included with the symbols of the positional markers to further identify the desired recording positions. Alternatively, text designations of the cardiac testing locations (left ventricular valve, right atrial valve, etc.) may be included. A pull-down menu allowing selection of anatomical or cardiac testing locations may be included to allow user selection of the preferred recording site designations. The same set of recording site designations are desirably used in the voice guided protocol.

During use of the exemplary system, as the voice guided protocol directs the user to each recording position in step 202, the positional markers 402, 404, 406, and 408 may be highlighted, one at a time, in sequence, according to the predetermined protocol. Thus, the user may be visually reminded of the desired placement of the stethoscope and the order in which to proceed with auscultation. By this exemplary means, the user may be desirably shown the anatomical position of the recording position at which heart sounds are currently being recorded.

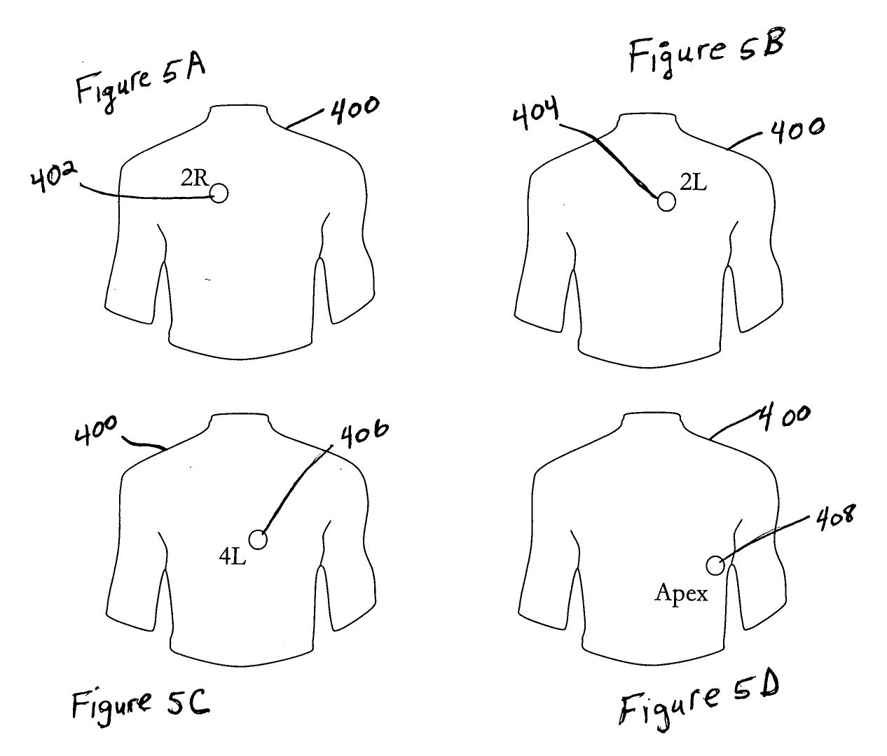

In an exemplary embodiment of the present invention, illustrated in FIGS. 5A-D, an auscultation protocol including four recording locations may be used. Exemplary graphical recording position map 308 of the graphical user interface desirably provides a visual representation of each recording position as heart sounds are recorded. Positional markers 402, 404, 406, and 408 are desirably highlighted one at a time in synchrony with the predetermined protocol. FIG. 5A illustrates the first recording position of the exemplary protocol. Positional marker 402, corresponding with the second intercostal space on the right (2R), is highlighted. FIGS. 5B, 5C, and 5D each illustrate a different highlighted positional marker to indicate the second, third and fourth recording positions in this protocol. Positional marker 404, in FIG. 5B, corresponds with the second intercostal space on the left (2L), positional marker 406, in FIG. 5C, corresponds with the fourth intercostal space on the left (4L), and positional marker 408, in FIG. 5D, corresponds with the apex of the heart (Apex).

In an exemplary embodiment, the predetermined protocol provided in step 200 may include information regarding desired patient posture and/or dynamic auscultatory maneuvers. This information may be included in the voice guided protocol. Posture and dynamic auscultatory maneuver information may also desirably be included in an additional section of exemplary testing procedure screen display 300, or may be included in the information display in status indicator 304. In another exemplary embodiment, a menu may be provided on exemplary testing procedure screen display 300 to enable the user to choose the position of each patient while heart sounds are being recorded. A similar menu may be provided for dynamic auscultatory maneuvers. In another exemplary embodiment, the graphical user interface may include a pull-down operating language menu. This operating language pull-down menu allows the user the option of choosing an operating language for the auscultatory diagnostic decision support system. Other menus may be included to allow the user to select a desired auscultatory protocol from a plurality of predetermined auscultatory protocols.

An exemplary embodiment of the present invention may include an auditory protocol to guide the user through the heart sound recording at a sequence of sites as well as providing a visual confirmation of the proper heart sound recording at each site. This voice guided protocol desirably introduces the auscultatory procedure, prompting the user to place the stethoscope at each of the sites in turn. The audio prompts may be transmitted to earpiece 34 of electronic stethoscope 31, or to speakers of general purpose computer 32 or other external speakers for guiding the user through the predetermined auscultatory protocol. The voice guided protocol may be produced from a pre-recorded voice track or may be generated by general purpose computer 32 using text-to-speech software.

Audio warnings may signal when recording of heart sounds at a recording position is to begin and end so that the user knows how long to record data from the recording position corresponding to each positional marker. During the period before recording begins at a new location the user may use amplification control 38 on electronic stethoscope 31 to adjust the heart sound to improve recording quality. Audio feedback may also be provided after each recording to alert the user to problems in the data quality found by the analysis device and request that the user re-record the data. This audio feedback may desirably be used to alert the user to problems such as the presence of unacceptable noise interference, signal clipping, or loss of signal. A final prompt desirably signals the completion of the protocol and alerts the user when the analysis is complete. Additional prompts can be included in the protocol to address changes in posture, dynamic auscultatory maneuvers, etc.

In an alternative embodiment, the user may desirably play back each recorded heart sound signal to check the quality of the signal. The user may then choose to re-record the data at any location where the recorded data is deemed unacceptable. Additionally, audio feedback may be provided after heart sound recordings where the data is identified as being suspect by the system. If problems are identified in the data quality, such as unacceptable noise levels due to motion artifact or background speech, signal clipping from gain setting problems, loss of signal due to stethoscope time out, etc., the voice guided protocol may be triggered and the user instructed to re-record the data.

Once the predetermined protocol is completed, the recorded heart sound signals are analyzed to determine auscultatory findings, step 206, which may be used to assist in making a diagnostic decision. This analysis is desirably carried out by general purpose computer 32 instructed by any one of a number of analysis programs, such as the multi-modal cardiac diagnostic decision support method disclosed in U.S. Pat. No. 6,572,560, “Multi-modal Cardiac Diagnostic Decision Support System and Method,” to Watrous et al. It is also contemplated that this analysis could be performed by special purpose heart sound signal processing circuitry. Alternatively, preprocessing of the heart sound signals may be performed by signal processing circuitry and further analysis carried out using cardiac signal diagnostic software.

The analysis of the heart sounds may desirably result in the identification of standard auscultatory findings (e.g. late-systolic murmur of grade III, mid-systolic click, loud S2 with wide, fixed splitting, etc.) within the heart sound signals. These standard auscultatory findings may be familiar to the physician, and, therefore, their determination may enable the physician to integrate the results of the heart sound analysis readily with other patient information. Certain hemodynamic parameters may also be derived from the acoustic signals (e.g. heart rate, HRV, systolic/diastolic durations). The analysis of the recorded heart sound signals in step 206 may further include analyzing the heart sounds for a presence of murmurs.

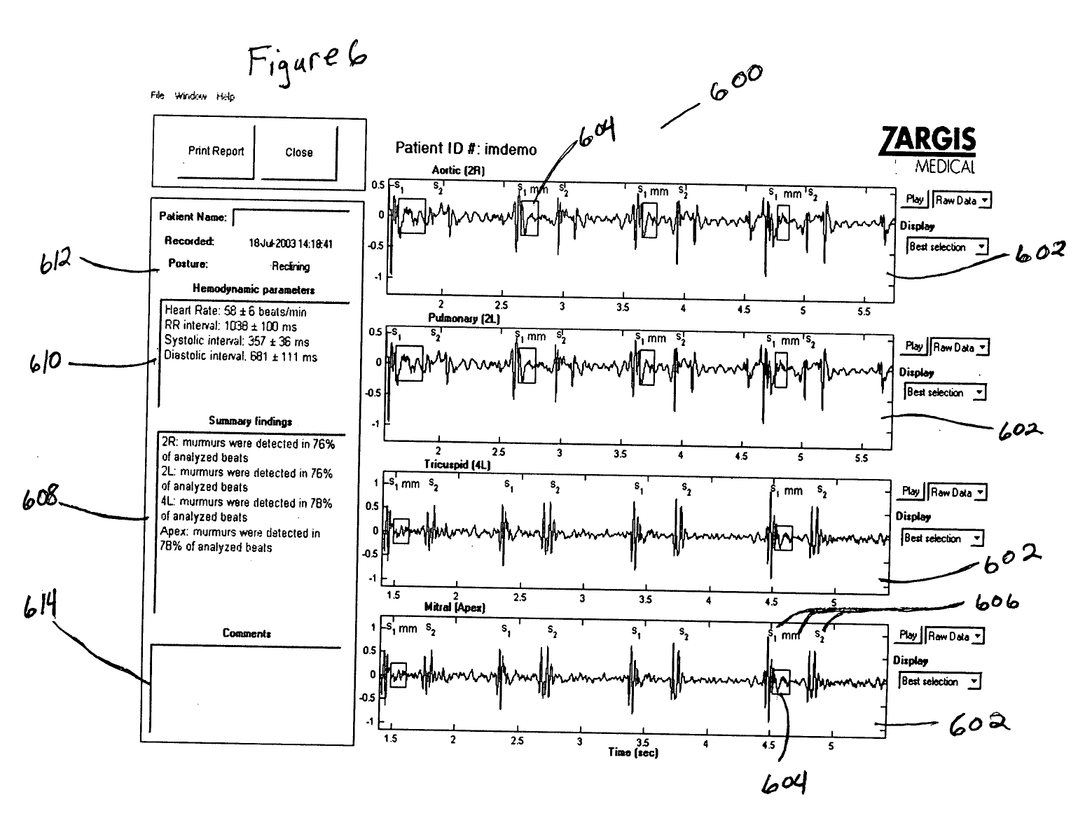

Exemplary analyzed heart sound signals and auscultatory findings from the analysis of the recorded heart sound signals are desirably displayed, step 208, to assist in making auscultatory diagnostic decisions. In this exemplary embodiment of the present invention, illustrated in FIG. 6, the analyzed heart sound signals and auscultatory findings may be displayed in a single window as shown in exemplary result screen display 600. Alternatively, the analyzed heart sound signals and auscultatory findings may be separated into separate display windows or may be accessible through a menu. The display of the analyzed heart sound signals and auscultatory findings may involve printing this information, either in conjunction with, or in place of, providing a result screen display.

Exemplary result screen display 600 desirably includes analyzed heart sound traces 602, summary findings section 608, hemodynamic parameter section 610, identification section 612, and comments section 614. These sections are not meant to be exhaustive, but are exemplary of a desirable organization of the auscultatory findings.

Exemplary analyzed heart sound traces 602 display the analyzed heart sounds in a graphical manner. A selected sequence of several heartbeats may be extracted from the longer (10-20 second) recording for each recording position and presented visually. These excerpts are desirably displayed horizontally and organized vertically by recording site so that they can be easily compared. The displays are desirably aligned on the left by the first heart sound, so that the cardiac cycle timings can be compared at various sites. These traces may also desirably include a compilation of results of signal processing and an analysis of the recorded heart sounds, which may be displayed graphically. Heart sounds detected in step 206 by the analysis algorithms (e.g. S1, S2, S3, ejection clicks, etc.) may be annotated by heart sound labels 606 at corresponding points on analyzed heart sound traces 602. These heart sound labels may be textual abbreviations as shown in FIG. 6, or they may be icons or a combination thereof. Murmurs detected in step 206 by the analysis algorithm may also be annotated and/or highlighted by transparent or semi-transparent boxes 604 that may desirably approximate the time-amplitude contour of the murmur. These annotations provide a graphical presentation of the analysis results which may desirably be used to confirm the correctness of the analysis, as well as the summary clinical findings displayed in summary findings section 608.

Furthermore, displaying the compilation of results may include the step of textually describing auscultatory findings in summary findings section 608. The results of the analysis of the heart sounds may be presented in summary textual form in terms of standard auscultatory findings (e.g. late-systolic murmur of grade III, mid-systolic click, loud S2 with wide, fixed splitting, etc.) in summary findings section 608. These standard auscultatory finding terms are desirably familiar to physicians, and, therefore, may enable physicians to readily integrate the results of the heart sound analysis readily with other patient information. Thus, summary auscultatory findings expressed in terms of standard clinical auscultatory terms used by physicians may desirably simplify the use of these summary finding in making diagnostic and referral decisions. A listing of possible cardiovascular diseases, consistent with the derived auscultatory findings, may also be included. This information may further assist the physician in associating clinical findings with various diseases.

Certain hemodynamic parameters may be derived in step 206 from the recorded cardiac acoustic signals (e.g. heart rate, HRV, systolic/diastolic durations). These hemodynamic parameters are desirably presented in numerical form, with mean/variance values in hemodynamic parameter section 610. Hemodynamic parameter section 610 may also include an indication of the presence of any irregularity in heart rhythm.

Identification section 612 includes identification information to identify the auscultatory test result. This identification information may also be used to associate the auscultatory finding with other patient data that may be in the computer system to allow more thorough diagnostic decision support. Exemplary results screen display 600 may also include comments section 614, which may include user comments, or information about signal quality, including the taking of retests during step 204.

Although the invention is illustrated and described herein with reference to specific embodiments, the invention is not intended to be limited to the details shown. Rather, various modifications may be made in the details within the scope and range of equivalents of the claims and without departing from the invention. Specifically, it is contemplated that, although the present invention has been described in terms of auscultation for cardiac diagnostic decision support, one skilled in the art may understand that the present invention may also be used in conjunction with auscultation focusing on the pulmonary or gastrointestinal diagnostic decision support. Furthermore, the present invention may be applicable to signal acquisition and conditioning, or filtering, to produce a display of heart sound signals (e.g. an enhanced phonocardiogram), by which relevant signal characteristics may be enhanced and readily viewed.

What is claimed:

1. A method for detecting a plurality of heart sounds in an auscultatory process comprising the steps of:
providing a predetermined protocol corresponding with a plurality of recording locations on a patient;
instructing a user to follow the predetermined protocol for recording the plurality of heart sounds at the plurality of recording locations using at least one of a voice guided protocol and a graphical user interface; and
detecting and recording the heart sounds according to the predetermined protocol.

2. The method according to claim 1 wherein the step of detecting and recording the heart sounds includes detecting the plurality of heart sounds using a noninvasive passive acoustic sensor to detect heart sounds from well-defined and standard positions on a chest surface.

3. The method according to claim 1 further comprising the step of displaying the recorded heart sounds in a graphical manner.

4. The method according to claim 1 further comprising the step of automatically analyzing the recorded heart sounds to determine auscultatory findings.

5. The method according to claim 4 further comprising the step of displaying a compilation of results of the analyzed heart sounds in a graphical manner.

6. The method according to claim 5 wherein the step of displaying the compilation of results further comprises the step of textually describing the determined auscultatory findings.

7. The method according to claim 6 wherein the auscultatory findings are described in terms of standard clinical auscultatory findings used by physicians to make diagnostic and referral decisions.

8. The method according to claim 5 further comprising analyzing the heart sounds for a presence of murmurs.

9. The method according to claim 1 further wherein the step of detecting and recording the heart sounds further includes prompting the user to re-record one of the plurality of heart sounds when the one heart sound is determined to include an error.

10. The method according to claim 1 further comprising the step of displaying a prompt to allow a user to override the predetermined protocol.

11. A computer readable medium adapted to instruct a general purpose computer to detect a plurality of heart sounds in an auscultatory process, the method for comprising the steps of:
providing a predetermined protocol corresponding with a plurality of recording locations on a patient;
instructing a user to follow the predetermined protocol for recording the plurality of heart sounds at the plurality of recording locations using at least one of a voice guided protocol and a graphical user interface; and
detecting and recording the heart sounds according to the predetermined protocol.

12. An auscultatory diagnostic decision support system comprising:
a cardiac acoustic sensor to produce a heart sound signal;
a heart sound analysis device adapted to receive and analyze the heart sound signal; and
a display device including a graphical user interface (GUI) to guide a user through a predetermined protocol.

13. The apparatus according to claim 12 further comprising means for transmitting the heart sound signal to the heart sound analysis device by at least one of a wire, an infrared signal, and a wireless signal.

14. The apparatus according to claim 12 wherein the GUI includes at least one of:
a pull-down menu having a plurality operating languages for selecting an operating language of the auscultatory diagnostic decision support system;
a pull-down menu having a plurality of auscultatory protocols for selecting the predetermined auscultatory protocol; and
a pull-down menu having a plurality of recording site designations for selecting a recording site designation of the auscultatory diagnostic decision support system.

15. The apparatus according to claim 12 further comprising an earpiece wherein the GUI includes an re-record option to allow a user to interrupt the predetermined protocol and have the heart sound analysis device receive a second heart sound signal.

16. The apparatus according to claim 12 wherein the heart sound analysis device includes at least one of:
a general purpose computer;
special purpose circuitry; and
an application specific integrated circuit.

17. The apparatus according to claim 12 wherein the GUI includes a visual representation of an anterior thorax to guide the user and a plurality of positional markers to pinpoint desired placements of the cardiac acoustic sensor on the anterior thorax.

18. The apparatus according to claim 12 further comprising;
a speaker coupled to the display device; and
at least one of a pre-recorded voice track and text-to-speech software to generate audio signals;
wherein the audio signals are transmitted by the by the speaker as a series of audio prompts to guide the user through the predetermined auscultatory protocol.

19. The apparatus according to claim 12 wherein the cardiac acoustic sensor is an electronic stethoscope.

20. The apparatus according to claim 19 further comprising;
at least one of a pre-recorded voice track and text-to-speech software to generate audio signals;
wherein the audio signals are transmitted by an earpiece of the electronic stethoscope as a series of audio prompts to guide the user through the predetermined auscultatory protocol.

21. A user interface for an auscultatory diagnostic decision support system comprising:
a graphical user interface (GUI) to guide a user of the auscultatory diagnostic decision support system through a predetermined auscultatory protocol including;
a visual representation of a body portion of a patient;
a plurality of positional markers to pinpoint a plurality of auscultatory measurement locations on the body portion; and
a visual presentation of a measured acoustic signal corresponding to each auscultatory measurement location; and
a speaker to provide a voice guided protocol including a series of audio prompts to guide the user through the predetermined auscultatory protocol.

22. The interface according to claim 21 wherein the speaker is at least one of:
an earpiece of an electronic stethoscope; and
a speaker of a general purpose computer used to display the GUI.

23. The interface according to claim 21 wherein the series of audio prompts identify the plurality of auscultatory measurement locations in an order representing the predetermined auscultatory protocol.

24. The interface according to claim 21 wherein the series of audio prompts include at least one of:
a posture prompt to identify a change in posture corresponding to at least one of the plurality of auscultatory measurement locations;
a completion prompt to identify completion of the predetermined auscultatory protocol; and
an auscultatory maneuver prompt to identify a dynamic auscultatory maneuver corresponding to at least one of the plurality of auscultatory measurement locations.

25. The interface according to claim 21 wherein the body portion of the patient included in the GUI is at least one of:
an anterior thorax;
a posterior thorax;
an anterior abdomen; and
a posterior abdomen.

26. A method for detecting a plurality of bodily sounds in an auscultatory process comprising the steps of:
providing a predetermined protocol corresponding with a plurality of recording locations on a patient;
instructing a user to follow the predetermined protocol for recording the plurality of bodily sounds at the plurality of recording locations using at least one of a voice guided protocol and a graphical user interface; and
detecting and recording the bodily sounds according to the predetermined protocol.

27. The method of claim 26 wherein the plurality of bodily sounds include at least one of heart sounds, lung sounds, and gastrointestinal sounds.

28. The method of claim 26 further comprising the step of analyzing the recorded bodily sounds to determine auscultatory findings.

29. A computer readable medium adapted to instruct a general purpose computer to detect a plurality of bodily sounds in an auscultatory process, the method for comprising the steps of:
providing a predetermined protocol corresponding with a plurality of recording locations on a patient;
instructing a user to follow the predetermined protocol for recording a plurality of bodily sounds at a plurality of recording locations using at least one of a voice guided protocol and a graphical user interface; and
detecting and recording the bodily sounds according to the predetermined protocol.

30. An auscultatory diagnostic decision support system comprising:
an acoustic sensor configured to produce a bodily sound signal;
a bodily sound analysis device adapted to receive and analyze the bodily sound signal; and
a display device including a graphical user interface (GUI) to guide a user through a predetermined protocol to obtain a sequence of bodily sound signals for use by the bodily sound analysis device.
